(19) United States
(12) Patent Application Publication
(10) Pub. No.: US 2026/0335623 A1
(43) Pub. Date: Oct. 29, 2026

(54) FLUID DISPLACEMENT REGULATING VALVE

(71) Applicant: CareFusion 303, Inc., San Diego, CA (US)

(72) Inventors: Soon Yong PARK, Cypress, CA (US); Siddarth K. SHEVGOOR, Mission Viejo, CA (US); Kuochu Colin HUANG, Walnut, CA (US)

(21) Appl. No.: 19/642,255

(22) Filed: Apr. 8, 2026

Related U.S. Application Data
(60) Provisional application No. 63/794,218, filed on Apr. 24, 2025.

(51) Int. Cl. A61M 39/24; A61M 5/14

(52) U.S. Cl. CPC A61M 39/24; A61M 5/1413; A61M 2039/242; A61M 2039/2433

(57) ABSTRACT

Displacement regulating fluid valves described herein can be used in an intravenous (IV) delivery set to limit fluid displacement upon disconnection of a needleless fluid connector from a fluid source. A fluid displacement regulating valve may include an inlet couplable to a needleless fluid connector, an outlet couplable to the IV set, and a reservoir and a check valve positioned therebetween. The reservoir can store the portion of the fluid from the fluid source when a pressure at the reservoir is less than a pressure at the inlet and release the portion of the fluid towards the needleless fluid connector when the pressure at the reservoir exceeds the pressure at the inlet. The check valve permits the fluid to move from the reservoir to the outlet when the pressure at the reservoir is approximately equal to or greater than a cracking pressure of the check valve.

### RELATED APPLICATIONS

This application claims priority to U.S. Provisional Application No. 63/794,218, which was filed on Apr. 24, 2025, and is entitled “FLUID DISPLACEMENT REGULATING VALVE.”

### TECHNICAL FIELD

The present invention relates generally to needleless connectors and, in particular, to neutral fluid displacement regulating valves.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag.

In some instances, the IV set includes needleless fluid connectors to fluidly connect the IV catheter and the fluid source. These needleless fluid connectors often positively or negatively displace some of the fluid when they are disconnected from the fluid source. Some needleless fluid connectors are prone to positive displacement, in which the needleless fluid connector directs some of the fluid towards the patient upon disconnection. Some needleless fluid connectors are prone to negative displacement, in which the needleless fluid connector draws some of the fluid—and, in some cases, blood—out of the patient upon disconnection.

### SUMMARY

Fluid displacement regulating valves are disclosed that can include or be coupled to a needleless fluid connector. During priming of a fluid flow pathway coupled to the fluid displacement regulating valves, a reservoir in the fluid displacement regulating valve can become filled with a priming fluid from the fluid source, and a check valve in the fluid displacement regulating valve can cause the fluid to be retained in the reservoir while the fluid or another fluid is moved through the fluid displacement regulating valve. When the needleless fluid connector is disconnected from the fluid source, the fluid stored in the reservoir can be released or drawn into the needleless fluid connector to provide neutral fluid displacement.

In some implementations of the present disclosure, a displacement regulating fluid connector assembly is disclosed. The displacement regulating fluid connector assembly comprises a needleless fluid connector comprising a connector inlet, a connector outlet, and a connector body positioned between and fluidly connected to the connector inlet and the connector outlet, wherein the connector inlet is configured to couple to an intravenous (IV) set and permit fluid to flow from the IV set into the connector body; a displacement regulating valve comprising an inlet coupled to the connector outlet, an outlet, an inner surface forming at least a portion of a reservoir with a first opening fluidically coupled to the inlet, and a second opening fluidically coupled to the outlet; a membrane coupled to the inner surface to form another portion of the reservoir; a check valve fluidly connected between the reservoir and the outlet, wherein the check valve comprises a cracking pressure such that, when a fluid pressure at the reservoir is approximately equal to or greater than the cracking pressure, the check valve permits the fluid to move through the check valve from the reservoir to the outlet, and when the fluid pressure is below the cracking pressure, the check valve is configured to resist movement of the fluid from the reservoir toward the outlet such that at least a portion of the fluid remains in the reservoir.

In some embodiments of the present disclosure, a displacement regulating valve is configured to fluidly connect a first part of an IV set to a second part of the IV set, the displacement regulating valve comprising an inlet, and outlet, an inner surface forming at least a portion of a reservoir, the reservoir comprising a first opening fluidically coupled to the inlet and a second opening fluidically coupled to the outlet; a membrane coupled to the inner surface to form another portion of the reservoir; a check valve positioned between the reservoir and the outlet, the check valve configured to permit a fluid to flow from the reservoir to the outlet when a fluid pressure at the reservoir is approximately equal to or greater than a cracking pressure of the check valve, and the check valve configured to resist movement of the fluid from the reservoir toward the outlet when the fluid pressure is below the cracking pressure.

Some embodiments of the present disclosure provide a displacement regulating valve configured to fluidly connect a first part of an IV set to a second part of the IV set, the displacement regulating valve comprising a tube comprising a channel, a channel inlet, a channel outlet, and a reservoir aperture, wherein the tube is configured to receive a fluid from the first part of the IV set via the channel inlet; a reservoir that encloses the reservoir apertures; and a check valve comprising a check valve inlet coupled to the channel outlet and a check valve outlet configured to couple to the second part of the IV set, wherein, the check valve is configured to permit a fluid from to move from the first part of an IV set to the second part of the IV set when a fluid pressure at the check valve is approximately equal to or greater than a cracking pressure of the check valve, and the check valve configured to resist movement of the fluid toward the second part of the IV set when the fluid pressure is below the cracking pressure.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an intravenous (IV) set coupled to a patient, in accordance with aspects of the present disclosure.

FIG. 2 illustrates a displacement regulating fluid connector assembly comprising a needleless fluid connector and a fluid displacement regulating valve, in accordance with aspects of the present disclosure.

FIG. 3 illustrates a displacement regulating fluid connector assembly comprising a needleless fluid connector and another embodiment of a fluid displacement regulating valve, in accordance with aspects of the present disclosure.

FIG. 4A illustrates a cross-sectional side view of the fluid displacement regulating valve, taken from the line 4-4 in FIGS. 2 and 3, in accordance with aspects of the present disclosure.

FIG. 4B illustrates a perspective cross-sectional side view of the fluid displacement regulating valve of FIG. 4A, in accordance with aspects of the present disclosure.

FIG. 4C illustrates a partial cross-sectional view of the fluid displacement regulating valve of FIG. 4B with a membrane, in accordance with aspects of the present disclosure.

FIG. 4D illustrates a partially exploded view of the fluid displacement regulating valve and membrane shown in FIG. 4C with multiple channels, in accordance with aspects of the present disclosure.

FIG. 4E illustrates a partial cross-sectional view of the fluid displacement regulating valve of FIG. 4B with multiple channels, in accordance with aspects of the present disclosure.

FIG. 4F illustrates the fluid displacement regulating valve of FIGS. 4E with a tube extending through the reservoir, in accordance with aspects of the present disclosure.

FIG. 5 illustrates a displacement regulating fluid connector assembly comprising a needleless fluid connector and another embodiment of a fluid displacement regulating valve, in accordance with aspects of the present disclosure.

FIG. 6 illustrates a displacement regulating fluid connector assembly comprising a needleless fluid connector and another embodiment of a fluid displacement regulating valve, in accordance with aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

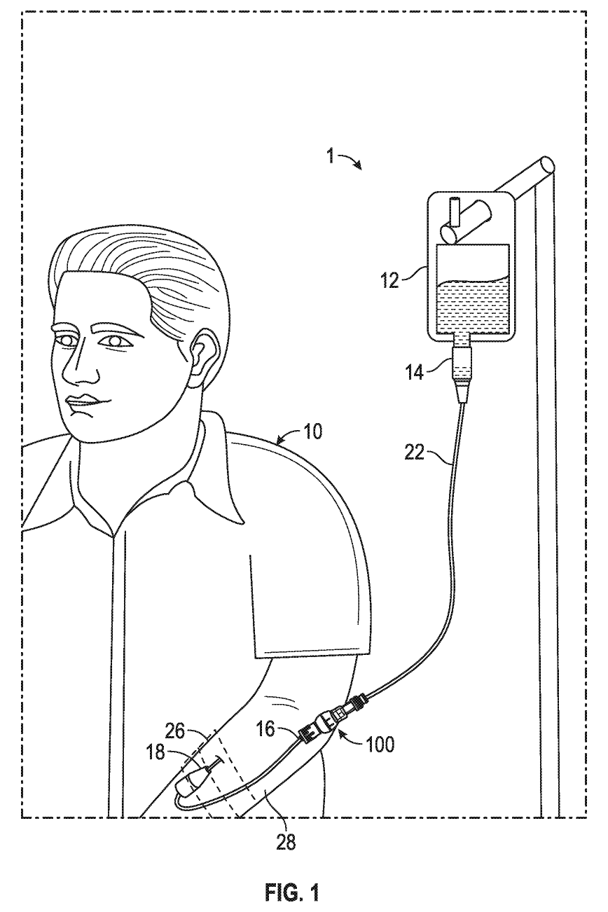

Referring now to the figures, FIG. 1 illustrates an intravenous (IV) set coupled to a patient, in accordance with aspects of the present disclosure. The IV set 1 includes a medical fluid bag 12 (also referred to as a fluid source 12), a drip chamber 14, a first tube 22, a displacement regulating fluid connector assembly 100, a second tube 16, a catheter 18, and tape 26. The tubing 22 extends between the drip chamber 14 and a needleless connector of the displacement regulating fluid connector assembly 100. The tubing 16 extends between a displacement regulating valve of the displacement regulating fluid connector assembly 100 and the catheter 18. The catheter 18 is inserted in the arm 28 of the patient 10; however, it should be understood that the present disclosure contemplates instance in which the displacement regulating valve of the displacement regulating fluid connector assembly 100 can be coupled to another portion of an IV set. The catheter 18 can be coupled to the patient by tape 26 placed over any of the tubing 16 and the catheter 18 to resist dislodgement of the catheter 18 or IV set from the patient.

The displacement regulating fluid connector assembly 100 can be used in medical applications, such as the IV set 1 as well as other medical fluid delivery applications, including peripheral intravenous catheters (PIVC), as non-limiting examples. In some embodiments of the present disclosure, the fluid connector assembly 100 includes a needleless fluid connector and a fluid displacement regulating valve. In other embodiments, the fluid connector assembly 100 includes a fluid displacement regulating valve that is couplable to a needleless fluid connector. In FIG. 1, the fluid connector assembly 100 is connected to tubing 22, but the fluid connector assembly 100 can also receive other fluid delivery devices such as a needleless syringe.

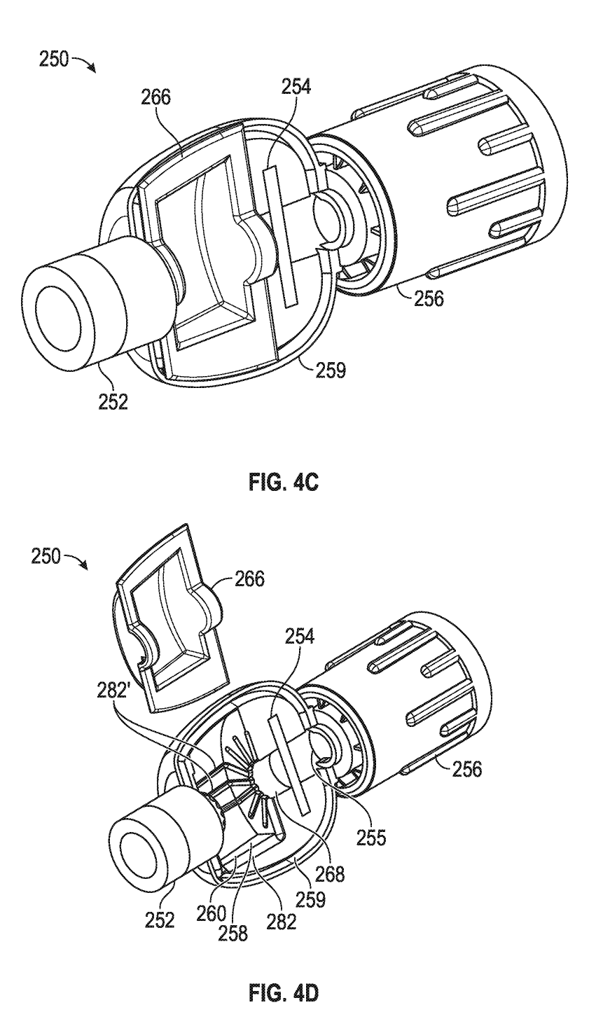

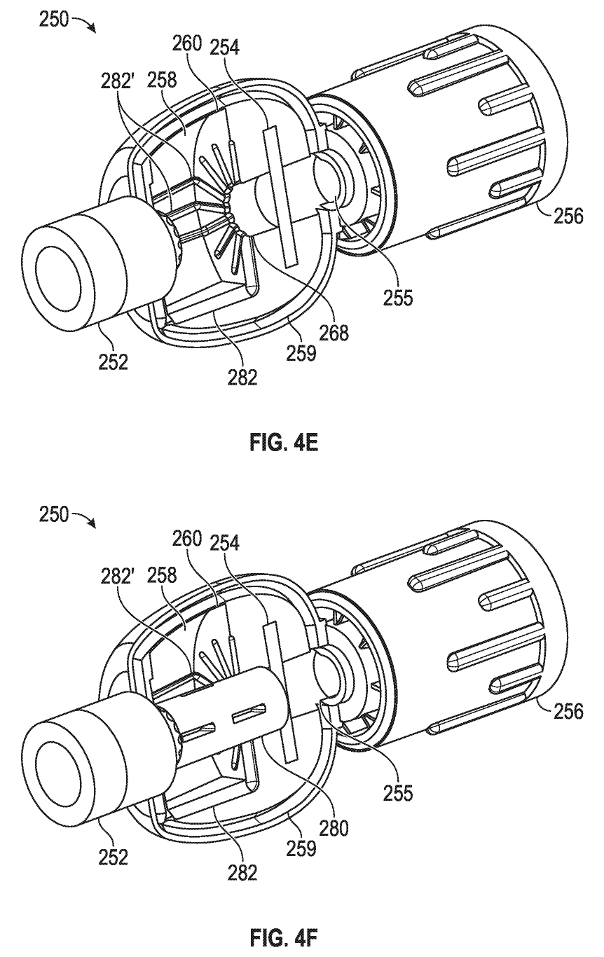

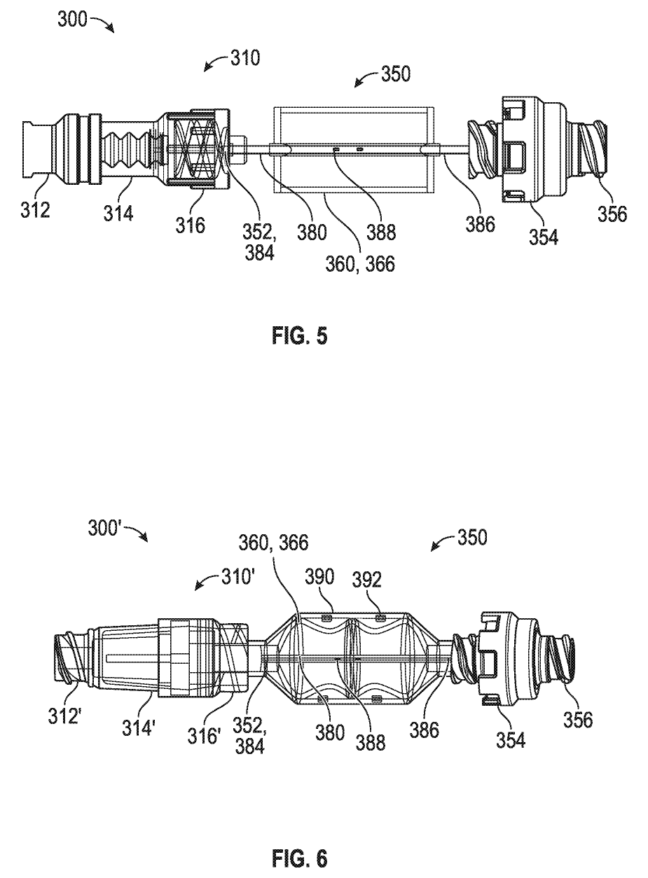

The fluid displacement regulating valve of the fluid connector assembly 100 can provide neutral displacement of a fluid by limiting or reducing positive and negative displacement of the fluid when the needleless fluid connector is disconnected from the tubing 22 (or otherwise disconnected from the fluid source 12). This is achieved, at least in part, due to the employment of a reservoir (e.g., the reservoir 260 in FIGS. 4A, 4B, and 4D-4F, and the reservoir 360 in FIGS. 5 and 6) within the fluid displacement regulating valve and a unidirectional or bidirectional check valve (e.g., the check valve 254 in FIGS. 4A-4F and the check valve 354 in FIGS. 5 and 6) that is fluidly connected to the reservoir. In the IV set 1 illustrated in FIG. 1, when the tubing 22, the fluid connector assembly 100, and the tubing 16 are being primed, the fluid fills the reservoir and, because of the check valve, a portion of the fluid from the fluid source 12 remains in the reservoir before, during, and after fluid delivery until the fluid connector assembly is disconnected from the fluid source 12. When the tubing 22 is disconnected from the fluid connector assembly 100 (or when the fluid source 12 is otherwise disconnected from the fluid connector assembly), the fluid in the reservoir is drawn out of the reservoir (i.e., in a direction away from the check valve or the patient 10, towards the tubing 22 or the fluid source 12, or “upstream”). By storing fluid in the reservoir that can be displaced upon disconnection, the displacement regulating valve can provide neutral displacement of fluid between the patient 10 and the IV set 1.

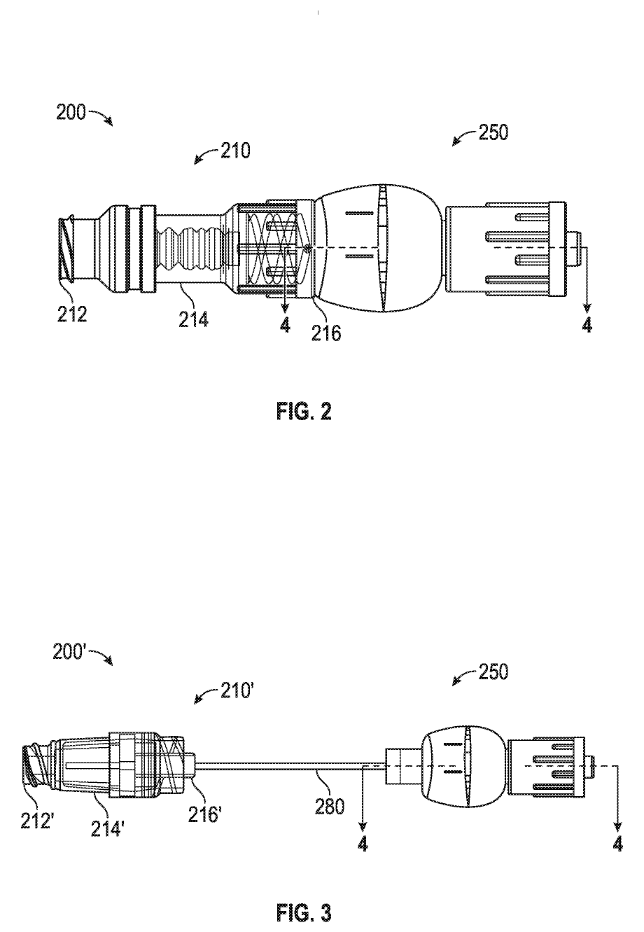

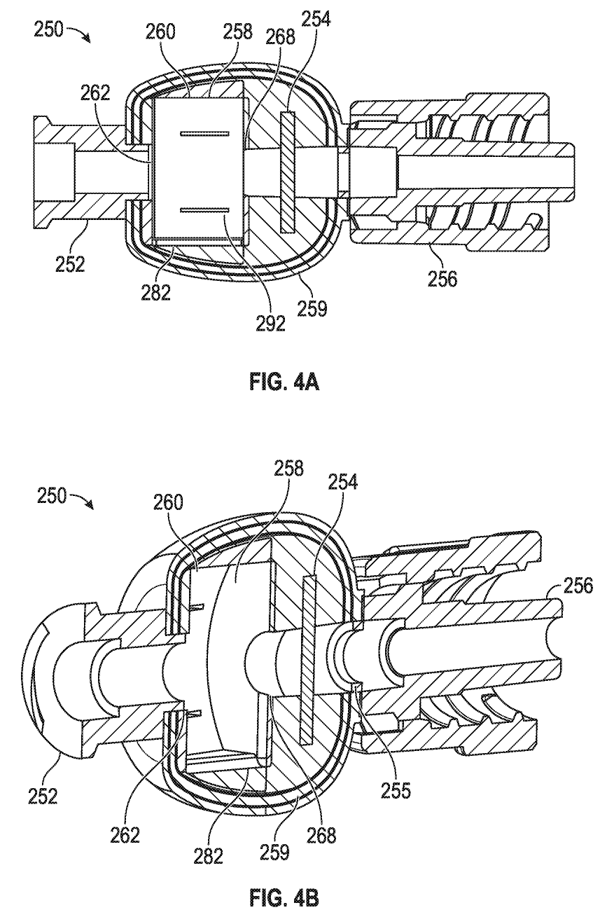

FIG. 2 illustrates a displacement regulating fluid connector assembly comprising a needleless fluid connector incorporated into a fluid displacement regulating valve, in accordance with aspects of the present disclosure. The displacement regulating fluid connector assembly 200 includes the same structure and functional benefits as the displacement regulating fluid connector assembly 100, except as described below. FIG. 2 depicts a displacement regulating fluid connector assembly 200 that includes a needleless fluid connector 210 integrated with a fluid displacement regulating valve 250. In FIG. 2, the needleless fluid connector 210 is a negative displacement needleless fluid connector, but other types of needleless fluid connectors 210 can also be integrated with the fluid displacement regulating valve 250. For example, the needleless fluid connector 210 can be a positive displacement needleless fluid connector (as shown by the needleless fluid connector 210’ in FIG. 3) or a neutral displacement needleless fluid connector.

The needleless fluid connector 210 includes a connector inlet 212, a connector body 214, and a connector outlet 216. The connector inlet 212 is fluidly connected to the connector outlet 216 by the connector body 214, which is positioned between the connector inlet 212 and the connector outlet 216. The connector inlet 212 is configured to couple to an IV set (e.g., the IV set 1 in FIG. 1 via the tubing 22, a syringe, or other fluid connector). The connector outlet 216 is couplable to the fluid displacement regulating valve 250, as will be described in greater detail with respect to FIGS. 4A and 4B.

The connector outlet 216 can be coupled to the fluid displacement regulating valve 250 by removably coupling the needleless fluid connector 210 and the fluid displacement regulating valve 250. The needleless fluid connector 210 and the fluid displacement regulating valve 250 can be removably coupled together by a thread on the fluid displacement regulating valve 250 that are configured to matingly engage another thread on the needleless fluid connector 210. It is also contemplated in the present disclosure that the needleless fluid connector 210 and the fluid displacement regulating valve 250 can be removably coupled together by any of an interference fit or mating luer features therebetween between. In some embodiments of the present disclosure, the needleless fluid connector 210 and the fluid displacement regulating valve 250 can coupled together by any of an adhesive of weld therebetween or the needleless fluid connector 210 and the fluid displacement regulating valve 250 can be unitarily formed together.

In some embodiments, the fluid displacement regulating valve 250 is at least partially made of a transparent or translucent material. For example, the fluid displacement regulating valve 250 can be made of any of a polycarbonate material and an acrylic material. A transparent or translucent material can help a user, such as a nurse or other caregiver, see that a fluid is moving through the fluid displacement regulating valve 250. The fluid displacement regulating valve 250 can be entirely transparent or translucent. In other embodiments, an inlet and/or an outlet are transparent or translucent, which enables the user to confirm that the fluid is flowing in the correct direction. In some embodiments of the present disclosure, the fluid displacement regulating valve comprises a material that is any of opaque, translucent, semi-opaque, and or transmissive.

FIG. 3 illustrates a displacement regulating fluid connector assembly comprising a needleless fluid connector and another embodiment of a fluid displacement regulating valve, in accordance with aspects of the present disclosure. FIG. 3 depicts a displacement regulating fluid connector assembly 200’ that includes a needleless fluid connector 210’ and a fluid displacement regulating valve 250, where the fluid displacement regulating valve 250 can be coupled to the needleless fluid connector 210’ via a tube 280, which will be described below.

In FIG. 3, the needleless fluid connector 210’ is a positive displacement needleless fluid connector, but other types of needleless fluid connectors 210’ can also be coupled to the fluid displacement regulating valve 250. For example, the needleless fluid connector 210’ can be a negative displacement needleless fluid connector (as shown by the needleless fluid connector 210 in FIG. 2) or a neutral displacement needleless fluid connector.

The needleless fluid connector 210’ includes a connector inlet 212’, a connector body 214’, and a connector outlet 216’. The connector inlet 212’ is fluidly connected to the connector outlet 216’ by the connector body 214’, which is positioned between the connector inlet 212’ and the connector outlet 216’. The connector inlet 212’ is configured to couple to an IV set (e.g., the IV set 1 in FIG. 1 via the tubing 22, a syringe, or other fluid connector). The connector outlet 216’ is couplable to the fluid displacement regulating valve 250, as will be described in greater detail with respect to FIGS. 4A and 4B.

While FIG. 2 depicts a fluid displacement regulating valve 250 as an integrated component of the displacement regulating fluid connector assembly 200, FIG. 3 demonstrates that a fluid displacement regulating valve 250 can also exist as a standalone component that can be coupled to a variety of needleless fluid connectors (or other fluid connector devices). In some embodiments, this can be accomplished via the tube 280 of the fluid displacement regulating valve 250. The tube 280 protrudes from the fluid displacement regulating valve 250 and, when the displacement regulating fluid connector assembly 200’ is assembled, the tube 280 is positioned between and fluidly connects the needleless fluid connector 210’ and the fluid displacement regulating valve 250. In some embodiments, the tube 280 extends through the inner volume of the fluid displacement regulating valve 250 (i.e., through the reservoir 260 illustrated in FIGS. 4A and 4B).

FIG. 4A illustrates a cross-sectional side view of the fluid displacement regulating valve 250, taken from the line 4-4 in FIGS. 2 and 3, in accordance with aspects of the present disclosure. The fluid displacement regulating valve 250 comprises an inlet 252, a reservoir 260, a check valve 254, and an outlet 256. The inlet 252 can receive the connector outlet 216, 216’ of a needleless fluid connector 210, 210’ or receive the tube 280 (FIG. 3) to fluidly coupled the fluid displacement regulating valve with the connector outlet 216, 216’. In some embodiments, the fluid displacement regulating valve 250 comprises the tube 280 extending distally from the inlet 252 and fluidly coupled to the reservoir.

The inlet 252 is fluidly connected to the reservoir 260 via a first opening 262 of the reservoir 260. In turn, the reservoir 260 is fluidly connected to the check valve 254 via the second opening 268 of the reservoir 260. The check valve 254 fluidly connects the reservoir 260 to the outlet 256 when a fluid pressure in the reservoir 260 meets or exceeds a cracking pressure of the check valve 254, at which point the check valve permits fluid flow from the reservoir 260 towards the outlet 256. The outlet 256 can couple the fluid displacement regulating valve 250 to the IV line (e.g., via the tubing 16 in FIG. 1, a syringe, or direct connector to the IV catheter 18 in FIG. 1). Accordingly, fluid flows through the fluid displacement regulating valve 250 via a flow path formed by the inlet 252, the reservoir 260, the check valve 254, and the outlet 256.

The fluid displacement regulating valve 250 has an inner surface 258 that extends between the inlet 252 and the outlet 256. A portion of the inner surface 258 forms the reservoir 260, which is a cavity or hollow region of the fluid displacement regulating valve 250. As described above with respect to FIG. 1, the reservoir 260 is configured to store or retain a portion of the fluid from the fluid source (e.g., the fluid source 12 in FIG. 1). The portion of the fluid stored in the reservoir can also be referred to as the reservoir fluid. When fluid is flowing from the needleless fluid connector 210, 210’ or the fluid source 12 through the fluid displacement regulating valve 250 along the flow path described above, the reservoir 260 remains, at least partially, full of the reservoir fluid. When the fluid flow from the needleless fluid connector 210, 210’ or the fluid source stops, the reservoir fluid remains stored in the reservoir 260. When the needleless fluid connector 210, 210’ is disconnected from the fluid source (e.g., disconnected from the tubing 22 or otherwise disconnected from the fluid source 12 in FIG. 1), the reservoir fluid flows towards the inlet 252 and into the connector body 214, 214’ of the needleless fluid connector 210, 210’. This results from the pressure differential between the reservoir 260 and the inlet 252 (or between the reservoir 260 and the connector body 214, 214’). That is, the reservoir 260 stores the reservoir fluid when the fluid pressure in the reservoir 260 is less than a fluid pressure at the inlet 252 or in the connector body 214, 214’. The pressure difference, caused by disconnection of the needleless fluid connector 210, 210’ from the fluid displacement regulating valve 250, caused the reservoir fluid to be drawn towards the inlet 252, into the connector body 214, 214’. As a result, a negative displacement caused by the needleless fluid connector 210 is applied to the reservoir fluid instead of the fluid in the patient (or the patient’s blood). In some embodiments, all of the reservoir fluid flows into the connector body 214, 214’ to fill the connector body 214, 214’. In other embodiments, some of the reservoir fluid remains in the reservoir 260 after the connector body 214, 214’ is filled. In yet other embodiments, a small portion of the reservoir fluid can flow through the check valve 254 (discussed in greater detail, below) and through the outlet 256.

In some embodiments, at least a portion of the reservoir 260 is formed by a membrane 266, which is shown in FIGS. 4C and 4D. FIGS. 4C and 4D illustrate a partial cross-sectional and partially exploded views of the fluid displacement regulating valve in FIG. 4B with a membrane, in accordance with aspects of the present disclosure. The membrane 266 can be coupled to the inner surface 258 of the fluid displacement regulating valve 250 to form the reservoir 260 between the membrane 266 and the inner surface 258. The inner surface 258 can include grooves 259, and the membrane 266 can be coupled to the inner surface 258 via the grooves 259. The membrane 266 is more flexible than the inner surface 258 such that the membrane 266 can move toward or away from the inner surface 258, thereby providing a reservoir that can increase and decrease in volume. In some embodiments of the present disclosure, the membrane 266 is configured to stretch and contract to change the volume of the reservoir 260. In some embodiments, the membrane 266 can be configured with any of a bellows or a folded surface to permit the volume of the reservoir to increase or decrease.

In some embodiments of the fluid displacement regulating valve 250, the inner surface 258 of the fluid displacement regulating valve 250 may also include one or more channels 282. The channels 282 can serve as alternative flow paths through the fluid displacement regulating valve 250, such as when the membrane 266 is moved toward the inner surface 258 such that the volume of the reservoir 260 decreases and the reservoir 260 deflates. In cases where the reservoir 260 deflates—such that fluid cannot flow between the inlet 252 and the outlet 256 via the first opening 262, the reservoir 260, and the second opening 268—the channels 282 can limit occlusion by allowing the fluid to flow between the inlet 252 and the outlet 256 through the flow pathways formed by the channels 282. In some embodiments of the present disclosure, the channels 282 provide additional fluid flow paths between the inlet 252 and the outlet 256.

Although FIG. 4A depicts one channel 282 extending along the inner surface 258, from the first opening 262 to the second opening 268, it should be understood that the present disclosure contemplates embodiments in which the fluid displacement regulating valve comprises more than one channel—and, thus, more than one flow pathway. For example, the fluid displacement regulating valve 250 can include multiple channels 282’ along the inner surface 258, each of which can form a different flow pathway. Embodiments of the fluid displacement regulating valve 250 with multiple channels 282’ are shown in FIGS. 4D-4F. FIGS. 4E and 4F illustrate partial cross-sectional views of the fluid displacement regulating valve of FIG. 4B with multiple channels and a tube extending through the reservoir, in accordance with aspects of the present disclosure.

In other embodiments, the tube 280 (shown in FIGS. 3 and 4F) extends through the reservoir 260 and the flow pathway is formed by the lumen of the tube 280 instead of through the channels 282, 282’. In yet other embodiments, one or more tubes can extend through the reservoir 260 between the first and second openings 262, 268 to form flow pathways. The one or more internal tube can be connected to the inner surface 258 or supported by posts that extend from the inner surface 258. In embodiments where the flow pathways are defined by tubes (e.g., the tube 280 or one or more internal tubes), the tubes may comprise a material that is more rigid than the membrane 266 such that the channel 282 (and optionally the channels 282’) remain open to provide the fluid flow pathway(s) when the reservoir 260 deflates.

In some embodiments, the inner surface 258 of the fluid displacement regulating valve 250 comprises a vent hole 292 that extends through a thickness of a wall that makes up the fluid displacement regulating valve 250. The vent hole 292 can be any shape and, in FIGS. 4A and 4B, are depicted as slots. In FIGS. 4A and 4B, two vent holes 292 are positioned on the part of the inner surface 258 that defines the reservoir 260. Optionally, the vent hole 292 can be positioned on other parts of the flow path through the fluid displacement regulating valve 250. In some embodiments, the walls of the fluid displacement regulating valve 250 are thicker around the vent holes 292. In other embodiments, the walls are thinner around the vent holes 292.

In embodiments where the reservoir 260 is defined by an inner surface of the membrane 266, the vent hole 292 can help relieve or limit pressure build-up as the reservoir 260 inflates and deflates as the membrane 266 stretches and contracts. To help relieve or limit pressure build-up as the reservoir 260 inflates and deflates, the vent hole 292 can be positioned between an outer surface of the membrane 266 and extend to an outer surface of the fluid displacement regulating valve 250.

FIG. 4A also illustrates the placement of the check valve 254 between the reservoir 260 and the outlet 256. In some embodiments, the check valve 254 comprises a cracking pressure that is high enough to resist the flow of the reservoir fluid between the reservoir 260 and the outlet 256 when there is no fluid flowing from the needleless fluid connector 210, 210’ or the fluid source (e.g., the fluid source 12 in FIG. 1) and low enough to permit the flow of the reservoir fluid when there is fluid flowing from the needleless fluid connector 210, 210’ or the fluid source. That is, the cracking pressure of the check valve 254 is slightly higher than the fluid pressure in the reservoir 260 when it is full of reservoir fluid but there is no fluid flowing from the needleless fluid connector 210, 210’ or the fluid source. Similarly, the cracking pressure is slightly higher than the rebounding pressure of an elastic or compressible valve in the needleless fluid connector 210, 210’. In this way, the check valve 254 helps the reservoir 260 store reservoir fluid and neutralizes the fluid displacement caused by the needleless fluid connector 210, 210’. The check valve 254 can also limit positive displacement of the fluid between the needleless fluid connector 210, 210’ and the patient by restricting flow from the outlet 256 towards the reservoir 260.

In other embodiments, the check valve 254 has a cracking pressure that is low enough to permit a small portion of the reservoir fluid to pass through the check valve 254 towards the outlet 256 when the fluid source is disconnected from the needleless fluid connector 210, 210’. In these embodiments, the check valve 254 cracking pressure may still be high enough to prevent most or all or the reservoir fluid from passing through the check valve 254 towards the outlet 256.

In some embodiments, the check valve 254 is a unidirectional or one-way valve that restricts or prevents fluid from being drawn from the patient through the fluid displacement regulating valve 250. In other embodiments, the fluid displacement regulating valve 250 may be used in settings where drawing fluid from the patient is desirable, so the check valve 254 can be bidirectional. The bidirectional check valves 254 have a lower cracking pressure in a flow direction from the reservoir 260 to the outlet 256 (or towards the patient) and a higher cracking pressure in the opposite direction from the outlet 256 to the reservoir 260 (or away from the patient).

In some embodiments, the check valve 254 is molded or otherwise manufactured directly into the fluid displacement regulating valve 250, which can facilitate efficient production of the fluid displacement regulating valve 250 and the production of smaller fluid displacement regulating valves 250. In other embodiments, the check valve 254 is manufactured separately from the fluid displacement regulating valve 250, and the fluid displacement regulating valve 250 has a cavity that can receive the check valve 254.

In some embodiments, different types of valves are used in lieu of the check valve 254. For example, a gate valve or a globe valve can be positioned within the fluid displacement regulating valve 250. The check valve 254 can comprise a plurality of arches coupled to a ring to form a dome, where the ring is adjacent to the reservoir 260 and an apex of the dome is adjacent to the outlet 256. The check valve 254 can also comprise any of a disc or diaphragm shape with a slit/septum or a plurality of slits/septum that are sealed below the cracking pressure and permit fluid flow at or above the cracking pressure.

FIG. 4B illustrates a perspective view of the fluid displacement regulating valve cross-section in FIG. 4A, in accordance with aspects of the present disclosure. FIG. 4B shows the depth of the reservoir 260 and the placement of the flow path of the channel 282. As discussed above, the reservoir 260 may be defined by a flexible membrane 266 that can stretch and contract to change the volume of the reservoir 260, with the maximum volume being limited by the space defined by the portion of the inner surface 258 that define the reservoir 260. The membrane 266 stretches, and the reservoir 260 inflates, when the reservoir 260 receives the reservoir fluid. The reservoir 260 reaches its maximum volume when the reservoir 260 is full of the reservoir fluid. In some embodiments, the maximum volume of the reservoir 260 is greater than or equal to a volume of the connector body 214, 214’ of the needleless fluid connector 210, 210’. In some embodiments, the maximum volume of the reservoir 260 is between 30 µL and 40 µL. In some embodiments, the maximum volume of the reservoir 260 is at least 35 µL.

When the reservoir fluid is drawn into the connector body 214, 214’ as described above with respect to FIG. 4A, the flexible membrane 266 can contract, and the reservoir 260 can deflate. In some embodiments, the contraction of the membrane 266 applies a force to the reservoir fluid that helps drive the reservoir fluid towards the connector body 214, 214’ by squeezing the reservoir fluid out of the reservoir 260.

As mentioned above with respect to FIG. 4A and depicted in FIG. 4C, the membrane 266 can be coupled to the inner surface 258 via grooves 259. FIGS. 4B and 4D show that the grooves 259 can extend between the first opening 262 of the reservoir 260, which is adjacent to the inlet 252, and an entrance 255 to the outlet 256. The membrane 266 can extend across the reservoir 260 and the check valve 254. In other embodiments, the grooves 259 extend between the first opening 262 and the second opening 268, in which case the membrane 266 extends only across the reservoir 260.

In some embodiments, the membrane 266 is hermetically sealed when the fluid displacement regulating valve 250 is assembled. In other embodiments, the membrane 266 is coupled to the inner surface 258 via any of a sonic weld or an adhesive. In yet other embodiments, the membrane 266 is coupled to the inner surface 258 when opposing portions or walls of the displacement regulating valve 250 are coupled together.

In some embodiments, the membrane 266 is connected to the inner surface 258 by posts or other structures that extend from the inner surface 258. These posts can be used to generate turbulence within the reservoir 260, which can help flush the reservoir fluid out of the reservoir 260.

Referring now to FIG. 5, a displacement regulating fluid connector assembly 300 is shown comprising a needleless fluid connector and an embodiment of a fluid displacement regulating valve, in accordance with aspects of the present disclosure. The displacement regulating fluid connector assembly 300 includes the same structure and functional benefits as the displacement regulating fluid connector assemblies 100 and 200, except as described herein. FIG. 5 depicts a displacement regulating fluid connector assembly 300 that includes a needleless fluid connector 310 integrated with a fluid displacement regulating valve 350. Although the needleless fluid connector 310 is shown as a negative displacement needleless fluid connector, the present disclosure contemplate that other types of needleless fluid connectors can also be integrated with the fluid displacement regulating valve 350. For example, the needleless fluid connector can be a positive displacement needleless fluid connector, as shown by the needleless fluid connector 310’ in FIG. 6.

The needleless fluid connector 310 includes a connector inlet 312, a connector body 314, and a connector outlet 316. The connector inlet 312 is fluidly connected to the connector outlet 316 by the connector body 314, which is positioned between the connector inlet 312 and the connector outlet 316. The connector inlet 312 is configured to couple to an IV set (e.g., the IV set 1 in FIG. 1 via the tubing 22, a syringe, or other fluid connector). The connector outlet 316 is couplable to the fluid displacement regulating valve 350 via the inlet 352 of the fluid displacement regulating valve 350, which is the channel inlet 384 of the tube 380.

The fluid displacement regulating valve 350 comprises a tube 380 that has an inner surface that forms a lumen, flow pathway, or channel between a channel inlet 384 and a channel outlet 386. The tube 380 also has one or more reservoir apertures 388 on an outer surface of the tube 380 that extend into the flow pathway.

The tube 380 is surrounded by a membrane 366 with an inner surface that forms a reservoir 360. The membrane 366 envelopes or extends entirely around the tube 380, such that the reservoir is fluidly connected to the flow pathway via the reservoir apertures 388. When fluid is flowing from the needleless fluid connector 310 through the fluid displacement regulating valve 350, the reservoir 360 remains, at least partially, full of the reservoir fluid. When the fluid flow from the needleless fluid connector 310 or the fluid source stops, the reservoir fluid remains stored in the reservoir 360. When the needleless fluid connector 310 is disconnected from the fluid source (e.g., disconnected from the tubing 22 or otherwise disconnected from the fluid source 12), the reservoir fluid flows through the reservoir apertures 388, towards the inlet 352, and into the connector body 314. This is a response to the change in pressure between the reservoir 360 and the connector body 314 when the needleless fluid connector is disconnected from the fluid source. Like the reservoir 260 described above with respect to the fluid displacement regulating valve 250 in FIGS. 4A and 4B, the reservoir 360 stores the reservoir fluid when the fluid pressure in the reservoir 360 is less than a fluid pressure at the inlet 352 or in the connector body 314. The pressure difference draws the reservoir fluid towards the inlet 352, into the connector body 314, and away from the patient. As a result, the negative displacement caused by the needleless fluid connector 310 is applied to the reservoir fluid instead of the fluid in the patient (or the patient’s blood). In some embodiments, all of the reservoir fluid flows into the connector body 314 to fill the connector body 314. In other embodiments, some of the reservoir fluid remains in the reservoir 360 after the connector body 314 is filled. In yet other embodiments, a small portion of the reservoir fluid can flow through the check valve 354 (discussed in greater detail, below) and through the outlet 356.

Like the membrane described above with respect to FIGS. 4A-4D, the membrane 366 is configured to stretch or move outward as the reservoir 360 fills with reservoir fluid from the fluid source and the flow pathway. Similarly, the membrane 366 can contract or move inward to deflate the reservoir 360 as the reservoir fluid leaves the reservoir 360. The reservoir 360 reaches its maximum volume when the membrane 366 is completely stretched. In some embodiments, the maximum volume of the reservoir 360 is greater than or equal to a volume of the connector body 314 of the needleless fluid connector 310. In some embodiments, the maximum volume of the reservoir 360 is between 30 µL and 40 µL. In some embodiments, the maximum volume of the reservoir 360 is at least 35 µL.

When the reservoir fluid is drawn into the connector body 314, the reservoir 360 deflates as the flexible material of the membrane contracts or moves inward. In some embodiments, this contraction of the membrane applies a force to the reservoir fluid that helps drive the reservoir fluid into the flow pathway via the reservoir apertures 388, towards the channel inlet 384, and into the connector body 314 of the needleless fluid connector 310. While the membrane 366 may comprise a flexible material, the tube 380 may comprise a rigid material, relative to the material of the membrane. This reduces the likelihood of the flow pathway closing as the reservoir 360 deflates and, thus, reduces the likelihood of occlusions in the fluid displacement regulating valve 350.

In FIG. 5, the check valve 354 is coupled to the channel outlet 386 of the tube 380. In some embodiments of the fluid displacement regulating valve 350, the outlet 356 of the fluid displacement regulating valve 350 is also the outlet 356 of the check valve 354. The check valve 354 also comprises a cracking pressure that, like the check valve 254 described with respect to FIGS. 4A and 4B, is high enough to resist the flow of the reservoir fluid between the reservoir 360 and the outlet 356 when there is no fluid flowing from the needleless fluid connector 310 or the fluid source (e.g., the fluid source 12 in FIG. 1) and low enough to permit the flow of the reservoir fluid when there is fluid flowing from the needleless fluid connector 310 or the fluid source. Stated differently, the cracking pressure of the check valve 354 is somewhat higher than the fluid pressure in the reservoir 360 when it is full of reservoir fluid and there is no fluid flowing from the needleless fluid connector 310 or the fluid source. Likewise, the cracking pressure is somewhat higher than the rebounding pressure of an elastic or compressible valve in the needleless fluid connector 310. With this cracking pressure, the check valve 354 helps the reservoir 360 store reservoir fluid and neutralizes the fluid displacement caused by the needleless fluid connector 310 when it is disconnected from the fluid source. The check valve 354 can also limit positive displacement of the fluid between the needleless fluid connector 310 and the patient by restricting flow from the outlet 356 towards the reservoir 360.

In other embodiments, the check valve 354 has a cracking pressure that is low enough to permit a small portion of the reservoir fluid to pass through the check valve 354 towards the outlet 356 when the fluid source is disconnected from the needleless fluid connector 310. In these embodiments, the check valve 354 cracking pressure may still be high enough to prevent most or all or the reservoir fluid from passing through the check valve 354 towards the outlet 356.

In some embodiments, the check valve 354 is a unidirectional or one-way valve that restricts or prevents fluid from being drawn from the patient through the fluid displacement regulating valve 350. In other embodiments, the fluid displacement regulating valve 350 may be used in settings where drawing fluid from the patient is desirable, so the check valve 354 can be bidirectional. The bidirectional check valves 354 have a lower cracking pressure in a flow direction from the reservoir 360 to the outlet 356 (or towards the patient) and a higher cracking pressure in the opposite direction from the outlet 356 to the reservoir 360 (or away from the patient).

In some embodiments, different types of valves are used in lieu of the check valve 354. For example, a gate valve or a globe valve can be positioned within the fluid displacement regulating valve 350. The check valve 354 can comprise a plurality of arches coupled to a ring to form a dome, where the ring is adjacent to the reservoir 360 and an apex of the dome is adjacent to the outlet 356. The check valve 354 can also comprise a silicon disc with a slit/septum or a plurality of slits/septum that are sealed below the cracking pressure and permit fluid flow at or above the cracking pressure.

In some embodiments, the fluid displacement regulating valve 350 is at least partially made of a transparent or translucent material. For example, the tube 380 or the membrane 366 can be any of a transparent, translucent, semi-opaque, or transmissive material which can help the user see flow of fluid through the fluid displacement regulating valve 350. The entirety of the tube 380 or the entirety of the membrane 366 (or both) can be transparent or translucent. In other embodiments, only parts of the tube 380 or the membrane 366 are transparent or translucent. For instance, if the parts of the tube 380 near the channel inlet 384 and the channel outlet 386 are transparent, the user can confirm that a fluid is flowing therethrough. Optionally, part or all of the check valve 354 is transparent or translucent. The check valve 354 can be made of any of a polycarbonate material or an acrylic material.

FIG. 6 illustrates a displacement regulating fluid connector assembly 300’ comprising a needleless fluid connector 310’ and an embodiment of a fluid displacement regulating valve 350 having a housing 390. The needleless fluid connector 310’ is a positive displacement needleless fluid connector, but other types of needleless fluid connectors can also be coupled to the fluid displacement regulating valve 350. For example, the needleless fluid connector can be a negative displacement needleless fluid connector 310, as shown, for example in FIG. 5. The needleless fluid connector 310’ includes a connector inlet 312’, a connector body 314’, and a connector outlet 316’. The connector inlet 312’ is fluidly connected to the connector outlet 316’ by the connector body 314’, which is positioned between the connector inlet 312’ and the connector outlet 316’. The connector inlet 312’ is configured to couple to an IV set (e.g., the IV set 1 in FIG. 1 via the tubing 22, a syringe, or other fluid connector). Like the connector outlet 316 of the needleless fluid connector 310 in FIG. 5, the connector outlet 316’ is couplable to the fluid displacement regulating valve 350.

The housing 390 is positioned in between the inlet 352 and the check valve 354, and the housing 390 surrounds an outer surface of the membrane 366. The housing 390 comprises a material that is less flexible, relative to the membrane, so that the housing 390 can protect the reservoir 360 from high pressure use and externally applied forces such as user accidentally pressing on the membrane 366 or placing an object on the membrane 366. Moreover, the housing 390 can be, at least partially, any of translucent, transparent, semi-opaque, or transmissive. Therefore, like the fluid displacement regulating valve 250 and the check valve 354, the housing 390 can be made of any of a polycarbonate material or an acrylic material.

In some embodiments, the housing 390 comprises one or more vent holes 392. The vent holes 392 can be any shape and, in FIG. 6, are depicted as holes. The vent holes 392 can help relieve or limit pressure build-up as the reservoir 360 inflates and deflates as the membrane 366 stretches and contracts. In some embodiments, the walls of the housing 390 are thicker around the vent holes 392. In other embodiments, the walls are thinner around the vent holes 392.

The embodiments of the fluid displacement regulating valve 350 illustrated in FIGS. 5 and 6 can be a standalone component that is couplable to the needleless fluid connector 310, 310’ or be incorporated into the needleless fluid connector 310, 310’.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A displacement regulating fluid connector assembly, the assembly comprising: a needleless fluid connector comprising a connector inlet, a connector outlet, and a connector body positioned between and fluidly connected to the connector inlet and the connector outlet, wherein the connector inlet is configured to couple to an intravenous (IV) set and permit fluid to flow from the IV set into the connector body; a displacement regulating valve comprising: an inlet coupled to the connector outlet, an outlet, an inner surface forming at least a portion of a reservoir with a first opening fluidically coupled to the inlet, and a second opening fluidically coupled to the outlet; a membrane coupled to the inner surface to form another portion of the reservoir; a check valve fluidly connected between the reservoir and the outlet, wherein the check valve comprises a cracking pressure such that, when a fluid pressure at the reservoir is approximately equal to or greater than the cracking pressure, the check valve permits the fluid to move through the check valve from the reservoir to the outlet, and when the fluid pressure is below the cracking pressure, the check valve is configured to resist movement of the fluid from the reservoir toward the outlet such that at least a portion of the fluid remains in the reservoir.

Clause 2. The assembly of Clause 1, wherein the needleless fluid connector comprises a negative displacement fluid connector.

Clause 3. The assembly of Clause 1, wherein the needleless fluid connector comprises a positive displacement fluid connector.

Clause 4. The assembly of Clause 1, further comprising a tube positioned between the needleless fluid connector and the displacement regulating valve to fluidly connect the connector outlet to the inlet of the displacement regulating valve.

Clause 5. The assembly of Clause 1, wherein the displacement regulating valve comprises a material that is at least partially transparent.

Clause 6. The assembly of Clause 5, wherein the displacement regulating valve comprises a material that is any of a polycarbonate or an acrylic.

Clause 7. The assembly of Clause 1, wherein the displacement regulating valve comprises a vent hole.

Clause 8. The assembly of Clause 1, wherein a channel extends into the inner surface, forming a fluid flow pathway between the inlet and the outlet of the displacement regulating valve.

Clause 9. The assembly of Clause 1, wherein the membrane is hermetically sealed.

Clause 10. The assembly of Clause 1, wherein the inner surface of the displacement regulating valve comprises grooves, and the membrane is coupled to the inner surface via the grooves.

Clause 11. The assembly of Clause 1, wherein the membrane is coupled to the inner surface by either a sonic weld or an adhesive.

Clause 12. The assembly of Clause 1, wherein the membrane comprises a first material and the reservoir comprises a second material, the first material being more flexible relative to the second material.

Clause 13. The assembly of Clause 1, wherein the membrane is configured to stretch such that an inner volume of the reservoir increases when the reservoir receives the fluid from the inlet of the displacement regulating valve and the membrane is configured to contract such that the inner volume of the reservoir decreases to direct the fluid toward the connector inlet when the fluid pressure at the reservoir is greater than a fluid pressure at the connector body.

Clause 14. The assembly of Clause 13, wherein the inner volume of the reservoir increases when the membrane stretches and decreases when the membrane contracts, the inner volume being between a maximum volume and a minimum volume.

Clause 15. The assembly of Clause 14, wherein the maximum volume of the reservoir is greater than or equal to a volume of the connector body.

Clause 16. The assembly of Clause 14, wherein the maximum volume of the reservoir is at least 35 µL.

Clause 17. The assembly of Clause 1, wherein the reservoir is configured to store the portion of the fluid when a fluid pressure at the reservoir is less than a fluid pressure at the inlet of the displacement regulating valve and release the portion of the fluid towards the needleless fluid connector when the fluid pressure at the reservoir exceeds the fluid pressure at the inlet of the displacement regulating valve.

Clause 18. The assembly of Clause 1, wherein the check valve is a one-way check valve.

Clause 19. The assembly of Clause 1, wherein the fluid pressure reaches the cracking pressure when the reservoir is full and the fluid is flowing through the displacement regulating valve.

Clause 20. The assembly of Clause 1, wherein the fluid pressure is less than the cracking pressure when the reservoir is full and the fluid is not flowing through the displacement regulating valve.

Clause 21. The assembly of Clause 1, wherein the check valve comprises a plurality of arches coupled to a ring to form a dome.

Clause 22. The assembly of Clause 21, wherein the ring is adjacent to the reservoir and an apex of the dome is adjacent to the outlet.

Clause 23. A displacement regulating valve configured to fluidly connect a first part of an intravenous (IV) set to a second part of the IV set, the valve comprising: an inlet, and outlet, an inner surface forming at least a portion of a reservoir, the reservoir comprising a first opening fluidically coupled to the inlet and a second opening fluidically coupled to the outlet; a membrane coupled to the inner surface to form another portion of the reservoir; a check valve positioned between the reservoir and the outlet, the check valve configured to permit a fluid to flow from the reservoir to the outlet when a fluid pressure at the reservoir is approximately equal to or greater than a cracking pressure of the check valve, and the check valve configured to resist movement of the fluid from the reservoir toward the outlet when the fluid pressure is below the cracking pressure.

Clause 24. The valve of Clause 23, wherein the inlet is configured to fluidically coupled to a needleless fluid connector.

Clause 25. The valve of Clause 24, wherein the reservoir is configured to drive the fluid towards the needleless fluid connector when a fluid pressure at the reservoir exceeds the fluid pressure at the inlet.

Clause 26. The valve of Clause 23, wherein the inlet is configured to receive a tube.

Clause 27. The valve of Clause 23, wherein the displacement regulating valve at least partially comprises a transparent material.

Clause 28. The valve of Clause 23, wherein the displacement regulating valve comprises a vent hole.

Clause 29. The valve of Clause 23, wherein a channel extends into the inner surface, forming a fluid flow pathway between the inlet and the outlet.

Clause 30. The valve of Clause 23, wherein the inner surface of the displacement regulating valve comprises grooves, and the membrane is coupled to the inner surface via the grooves.

Clause 31. The valve of Clause 23, wherein the membrane comprises a first material and the reservoir comprises a second material, the first material being flexible relative to the second material.

Clause 32. The valve of Clause 23, wherein the membrane is configured to stretch when the reservoir receives the fluid from the inlet and contract to drive the fluid towards the first part of the IV set.

Clause 33. The valve of Clause 23, wherein the reservoir is configured to store a portion of the fluid when a fluid pressure at the reservoir is less than a fluid pressure at the inlet and release the portion of the fluid towards the first part of the IV set when the fluid pressure at the reservoir exceeds the fluid pressure at the inlet.

Clause 34. The valve of Clause 23, wherein the check valve comprises a plurality of arches coupled to a ring to form a dome.

Clause 35. A displacement regulating valve configured to fluidly connect a first part of an intravenous (IV) set to a second part of the IV set, the valve comprising an inlet configured to receive the first part of the IV set, a reservoir fluidly connected to the inlet, a check valve fluidly connected to the reservoir, and an outlet configured to receive the second part of the IV set, wherein the check valve is configured to limit negative displacement and the reservoir is configured to limit positive displacement.

Clause 36. A displacement regulating fluid connector assembly, the assembly comprising: a needleless fluid connector comprising a connector inlet, a connector outlet, and a connector body positioned between and fluidly connected to the connector inlet and the connector outlet, wherein the connector inlet is configured to couple to an intravenous (IV) set and permit fluid to flow from the IV set into the connector body; an displacement regulating valve comprising: a tube comprising an inner surface forming a channel extending between a channel inlet and a channel outlet, and a reservoir aperture extending between the channel and an outer surface of the tube, and wherein; a reservoir extending between the channel inlet and the channel outlet and defined by a membrane that surrounds the outer surface of the tube, wherein the reservoir is fluidly connected to the channel via the reservoir apertures; and a check valve comprising a check valve inlet coupled to the channel outlet and a check valve outlet configured to couple to the IV set and permit the fluid to flow from the reservoir to the IV set when a fluid pressure at the channel outlet reaches a cracking pressure of the check valve, wherein the channel inlet is coupled to the connector outlet such that the fluid from the needleless fluid connector can move through the channel to the check valve and the reservoir can retain a portion of the fluid.

Clause 37. The assembly of Clause 36, wherein the membrane is configured to stretch when the reservoir receives the fluid from the channel and contract when the connector inlet is uncoupled from the IV set, thereby driving the portion of the fluid towards the needleless fluid connector.

Clause 38. The assembly of Clause 37, wherein a volume of the reservoir is defined by the membrane, and the volume increases when the membrane stretches and decreases when the membrane contracts.

Clause 39. The assembly of Clause 36, further comprising a housing positioned in between the inlet and the check valve and surrounding the tube and the reservoir.

Clause 40. The assembly of Clause 39, wherein the membrane comprises a first material and the housing comprises a second material, the first material being flexible relative to the second material.

Clause 41. The assembly of Clause 39, wherein the housing comprises a translucent or transparent material.

Clause 42. The assembly of Clause 39, wherein the housing comprises vent holes.

Clause 43. The assembly of Clause 36, wherein the fluid pressure reaches the cracking pressure when the reservoir is full and the fluid is flowing through the displacement regulating valve.

Clause 44. The assembly of Clause 36, wherein the fluid pressure is less than the cracking pressure when the reservoir is full and the fluid is not flowing through the displacement regulating valve.

Clause 45. The assembly of Clause 36, wherein, when the reservoir releases the portion of the fluid, the assembly permits the portion of the fluid to flow into the channel via the reservoir aperture and into the connector body via the channel inlet and the connector outlet.

Clause 46. A displacement regulating valve configured to fluidly connect a first part of an intravenous (IV) set to a second part of the IV set, the displacement regulating valve comprising: a tube comprising a channel, a channel inlet, a channel outlet, and a reservoir aperture, wherein the tube is configured to receive a fluid from the first part of the IV set via the channel inlet; a reservoir that encloses the reservoir apertures; and a check valve comprising a check valve inlet coupled to the channel outlet and a check valve outlet configured to couple to the second part of the IV set, wherein, the check valve is configured to permit a fluid from to move from the first part of an intravenous (IV) set to the second part of the IV set when a fluid pressure at the check valve is approximately equal to or greater than a cracking pressure of the check valve, and the check valve configured to resist movement of the fluid toward the second part of the IV set when the fluid pressure is below the cracking pressure.

Clause 47. The valve of Clause 46, wherein the displacement regulating valve is configured to couple with a needleless fluid connector via the channel inlet, and the reservoir is configured to direct a fluid toward the needleless fluid connector when the first part of the IV set is disconnected from the channel inlet.

Clause 48. The valve of Clause 46, further comprising a housing positioned in between the inlet and the check valve and surrounding the tube and the reservoir.

Clause 49. The valve of Clause 46, wherein the check valve permits fluid flow out of the channel outlet when a fluid pressure upstream of the check valve inlet exceeds a cracking pressure of the check valve.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A displacement regulating fluid connector assembly, the assembly comprising:
a needleless fluid connector comprising a connector inlet, a connector outlet, and a connector body positioned between and fluidly connected to the connector inlet and the connector outlet, wherein the connector inlet is configured to couple to an intravenous (IV) set and permit fluid to flow from the IV set into the connector body;
a displacement regulating valve comprising:
an inlet coupled to the connector outlet, an outlet, an inner surface forming at least a portion of a reservoir with a first opening fluidically coupled to the inlet, and a second opening fluidically coupled to the outlet;
a membrane coupled to the inner surface to form another portion of the reservoir; and
a check valve fluidly connected between the reservoir and the outlet, wherein the check valve comprises a cracking pressure such that, when a fluid pressure at the reservoir is approximately equal to or greater than the cracking pressure, the check valve permits the fluid to move through the check valve from the reservoir to the outlet, and when the fluid pressure is below the cracking pressure, the check valve is configured to resist movement of the fluid from the reservoir toward the outlet such that at least a portion of the fluid remains in the reservoir.

2. The assembly of claim 1, further comprising a tube positioned between the needleless fluid connector and the displacement regulating valve to fluidly connect the connector outlet to the inlet of the displacement regulating valve.

3. The assembly of claim 1, wherein the displacement regulating valve comprises a material that is at least partially transparent.

4. The assembly of claim 1, wherein the displacement regulating valve comprises a vent hole.

5. The assembly of claim 1, wherein a channel extends into the inner surface, forming a fluid flow pathway between the inlet and the outlet of the displacement regulating valve.

6. The assembly of claim 1, wherein the inner surface of the displacement regulating valve comprises grooves, and the membrane is coupled to the inner surface via the grooves.

7. The assembly of claim 1, wherein the membrane is coupled to the inner surface by either a sonic weld or an adhesive.

8. The assembly of claim 1, wherein the membrane is configured to stretch such that an inner volume of the reservoir increases when the reservoir receives the fluid from the inlet of the displacement regulating valve and the membrane is configured to contract such that the inner volume of the reservoir decreases to direct the fluid toward the connector inlet when the fluid pressure at the reservoir is greater than a fluid pressure at the connector body.

9. The assembly of claim 8, wherein the inner volume of the reservoir increases when the membrane stretches and decreases when the membrane contracts, the inner volume being between a maximum volume and a minimum volume.

10. The assembly of claim 9, wherein the maximum volume of the reservoir is greater than or equal to a volume of the connector body.

11. The assembly of claim 9, wherein the maximum volume of the reservoir is at least 35 µL.

12. The assembly of claim 1, wherein the reservoir is configured to store the portion of the fluid when a fluid pressure at the reservoir is less than a fluid pressure at the inlet of the displacement regulating valve and release the portion of the fluid towards the needleless fluid connector when the fluid pressure at the reservoir exceeds the fluid pressure at the inlet of the displacement regulating valve.

13. The assembly of claim 1, wherein the check valve is a one-way check valve.

14. A displacement regulating valve configured to fluidly connect a first part of an intravenous (IV) set to a second part of the IV set, the displacement regulating valve comprising:
an inlet, and outlet, an inner surface forming at least a portion of a reservoir, the reservoir comprising a first opening fluidically coupled to the inlet and a second opening fluidically coupled to the outlet;
a membrane coupled to the inner surface to form another portion of the reservoir; and
a check valve positioned between the reservoir and the outlet, the check valve configured to permit a fluid to flow from the reservoir to the outlet when a fluid pressure at the reservoir is approximately equal to or greater than a cracking pressure of the check valve, and the check valve configured to resist movement of the fluid from the reservoir toward the outlet when the fluid pressure is below the cracking pressure.

15. The valve of claim 14, wherein the inlet is configured to fluidically coupled to a needleless fluid connector.

16. The valve of claim 15, wherein the reservoir is configured to drive the fluid towards the needleless fluid connector when a fluid pressure at the reservoir exceeds the fluid pressure at the inlet.

17. The valve of claim 14, wherein a channel extends into the inner surface, forming a fluid flow pathway between the inlet and the outlet.

18. The valve of claim 14, wherein the reservoir is configured to store a portion of the fluid when a fluid pressure at the reservoir is less than a fluid pressure at the inlet and release the portion of the fluid towards the first part of the IV set when the fluid pressure at the reservoir exceeds the fluid pressure at the inlet.

19. A displacement regulating valve configured to fluidly connect a first part of an intravenous (IV) set to a second part of the IV set, the displacement regulating valve comprising:
a tube comprising a channel, a channel inlet, a channel outlet, and a reservoir aperture, wherein the tube is configured to receive a fluid from the first part of the IV set via the channel inlet;
a reservoir that encloses the reservoir apertures; and
a check valve comprising a check valve inlet coupled to the channel outlet and a check valve outlet configured to couple to the second part of the IV set,
wherein, the check valve is configured to permit a fluid from to move from the first part of an intravenous (IV) set to the second part of the IV set when a fluid pressure at the check valve is approximately equal to or greater than a cracking pressure of the check valve, and the check valve configured to resist movement of the fluid toward the second part of the IV set when the fluid pressure is below the cracking pressure.

20. The valve of claim 19, wherein the displacement regulating valve is configured to couple with a needleless fluid connector via the channel inlet, and the reservoir is configured to direct a fluid toward the needleless fluid connector when the first part of the IV set is disconnected from the channel inlet.
